(19) United States
(12) Patent Application Publication
(10) Pub. No.: US 2014/0364771 A1
(43) Pub. Date: Dec. 11, 2014

(54) PRESSURE SENSITIVE ASSEMBLIES FOR LIMITING MOVEMENTS ADVERSE TO HEALTH OR SURGICAL RECOVERY

(71) Applicants: David A. Pitts, Naples, FL (US); Rachelle Yusufbekov, Cape Coral, FL (US); Jeffrey Bossert, Venice, FL (US); Adam Gourley, Auburndale, FL (US); Mollie C. Venglar, Fort Myers, FL (US)

(72) Inventors: David A. Pitts, Naples, FL (US); Rachelle Yusufbekov, Cape Coral, FL (US); Jeffrey Bossert, Venice, FL (US); Adam Gourley, Auburndale, FL (US); Mollie C. Venglar, Fort Myers, FL (US)

(21) Appl. No.: 14/278,822

(22) Filed: May 15, 2014

Related U.S. Application Data
(60) Provisional application No. 61/824,191, filed on May 16, 2013.

(51) Int. Cl. A61B 5/11; A61B 5/00; A61B 5/103

(52) U.S. Cl. CPC A61B 5/11; A61B 5/1036; A61B 5/6825; A61B 5/7282; A61B 5/7405; A61B 5/742; A61B 5/7455
USPC 600/595; 600/587

(57) ABSTRACT

Pressure sensitive devices, systems and methods for alerting a user of movements potentially adverse to health or surgical recovery are disclosed. The pressure sensitive device may include a force sensor placed along the anterior aspect of a hand; and a vibration motor in communication with the force sensor in close proximity to the user, e.g., along the posterior aspect of the wrist. The vibration motor is configured to vibrate upon the measured force exceeding a predetermined threshold. This threshold can be adjusted according to clinical application and/or user need. The pressure sensitive device may further include a memory chip or wireless transmitter for recording and relaying data associated with a patient profile, and is enabled to interface with a Wi-Fi sensing technology. Logged data may be used for patient rehabilitation.

### CROSS-REFERENCE TO RELATED APPLICATION(S)

The present application claims priority benefit to a provisional patent application entitled “Pressure Sensitive Glove for Limiting Movements Adverse to Surgical Recovery,” which was filed on May 16, 2013 and assigned Ser. No. 61/824,191. The entire content of the foregoing provisional patent application is incorporated herein by reference.

### TECHNICAL FIELD

The present disclosure generally relates to biomedical assemblies or systems for alerting the user and/or third parties when movements adverse to health and/or surgical recovery are performed. More specifically, the present disclosure relates to assemblies, e.g., pressure sensitive devices, that are adapted to be associated with a desired anatomical region/location and that measure the force applied thereto. For example, a pressure sensitive device may be positioned at the palm region of the hand and, based upon threshold force criteria, respond to certain force levels by providing a signal, e.g., an alarm, to notify the user and/or third party once the threshold criteria is met or exceeded.

### BACKGROUND

Post-operative management has a vital role in the successful rehabilitation of patients. Numerous surgical procedures, in addition to injuries that may not require surgical intervention, involve post-operative management including range of motion limitations and/or restrictions. For example, open-heart surgery, Caesarian sections, abdominal surgery, repair of fractured bones, joint replacements such as total hip or shoulder, tendon reconstruction, etc., each dictate post-operative limitations and restrictions of specific movements. The post-operative restrictions may function to reduce strain on replacement joints, and reconstructed bones, tendons and/or ligaments, as well as to prevent rupture and/or reopening of surgical incisions, e.g., from open-heart surgery and Caesarian sections.

During open-heart surgery, the sternal bone is cut vertically to gain access to the heart. After the surgery has been performed, the two halves of the chest are secured, allowing the sternum to heal. This healing process can take several months. During that time, the patient must restrict movements to avoid complications, including catastrophic rupture of the sternum. After open-heart surgery, the patient's mental state can be temporarily impaired for many reasons, including the effects of anesthesia, medications, and metabolic changes. These changes in mental state make it especially difficult to alter common motor patterns post surgery.

Moving from a sit-to-stand (STS) position using the assistance of the hands is a common restricted movement after open-heart surgery. Additionally, a STS movement generates a large amount of force through the pectoralis muscle group, placing the healing sternal bones and incision site under unsafe loads and increasing the patent's risk of complications.

Cardiothoracic harnesses have been developed to reduce sternal instability at an incision site if used immediately after surgery and during rehabilitation (www.aztecheart.com; www.hearthugger.com). More specifically, post-surgical support harnesses are intended to stabilize the sternum and bony thorax during recovery. Heart or cardiac pillows are also employed post-surgery to aid in a patient's recovery process. The heart/cardiac pillow is generally used to splint the fracture in the sternum when a patient moves or breathes in a way that could cause problems (www.therapeuticpillows.com). Of note, however, heart/cardiac pillows rely on a patient's attentiveness, which is commonly deficient in post-operative patients, e.g., patients with postoperative confessional state or delirium and in the case of dementia patients.

Additional surgical procedures and/or health conditions give rise to a need to moderate or control patient movements. For example, a range of abdominal surgeries, including Caesarian sections, raise post-surgical issues and concerns relative to patient movement and the restriction of forces applications, which may affect the surgical region.. Similarly, surgical procedures and health conditions that implicate muscle, ligament, and tendon issues can also require careful attention to patient movements and/or forces applied to the applicable anatomical region.

Despite efforts to date, there exists a need to remind patients to avoid high-risk movements during recovery and/or with respect to health conditions. For example, it would be advantageous to remind a patient not to use the assistance of the hands when engaging in STS movement. Additionally, it would be advantageous to alert caregivers and family members to attend to a patient when the patient is attempting to stand without adherence to “sternal precautions.”

These and other needs are addressed by the apparatus, systems, and methods of the present disclosure. Indeed, it is contemplated that the disclosed apparatus, systems, and methods may prove useful in addressing other problems and deficiencies in a number of technical areas. Therefore, the disclosed invention should not be construed as limited to addressing any of the particular problems or deficiencies discussed herein.

### BRIEF SUMMARY

According to an exemplary embodiment of the present disclosure, a device for limiting movement adverse to health or surgical recovery is provided. The device includes a wearable housing; at least one sensor disposed with respect to the wearable housing and configured to detect a pressure value; an indicator component configured to generate an alert; and a control circuit electronically coupled to the at least one sensor and the indicator component, wherein the control circuit is configured to activate the indicator component when the pressure value exceeds a predefined threshold. The predefined threshold may be advantageously established to forestall and/or limit movements that are potentially adverse to health or surgical recovery.

According to an additional exemplary embodiment of the present disclosure, a system for limiting movement adverse to health or surgical recovery is provided. The system includes a sensor configured to detect a pressure value; an indicator component configured to generate an alert; a processing unit electronically coupled with the sensor and the indicator component, and having a wireless module for transmitting and receiving wireless communications; and a power source. The processing unit is configured to activate the indicator component when the pressure value exceeds a predefined threshold. The predefined threshold may be advantageously established to forestall and/or limit movements that are potentially adverse to health or surgical recovery. The wireless module wirelessly transmits the pressure value for data storage.

According to a further exemplary embodiment of the present disclosure, a method of detecting movements potentially adverse to health or surgical recovery is provided. The method includes detecting a force applied to a flexible sensor; outputting a pressure value of the force to a processing unit; and signaling an indicator component to generate an alert if the pressure value exceeds a predefined threshold.

Additional advantageous features, functions and applications of the disclosed devices, systems and methods will be apparent from the description which follows, particularly when read in conjunction with the appended figures.

### BRIEF DESCRIPTION OF THE DRAWINGS

The drawings presented herein are for illustration purposes only and are not intended to limit the scope of the present disclosure in any way. For a fuller understanding of the present disclosure, reference should be made to the following detailed description, taken in connection with the accompanying drawings, in which:

FIG. 1 is a perspective view of a human torso;

FIG. 2A is a top view of a pressure sensitive device according to an exemplary embodiment of the present disclosure;

FIG. 2B is a perspective view of the exemplary pressure sensitive device of FIG. 2A;

FIG. 2C is an end view of the exemplary pressure sensitive device of FIG. 2A;

FIG. 2D is a side view of the exemplary pressure sensitive device of FIG. 2A;

FIG. 3 is a front view of a pressure sensitive device according to an exemplary embodiment of the present disclosure;

FIG. 4 is a front-side view of a pressure sensitive device according to an exemplary embodiment of the present disclosure;

FIG. 5 is a bottom view of the pressure sensitive device showing the anterior aspect of the wrist according to an exemplary embodiment of the present disclosure;

FIG. 6 depicts a pressure sensitive device positioned on a patient hand according to an exemplary embodiment of the present disclosure;

FIG. 7 depicts a pressure sensitive device according to an exemplary embodiment of the present disclosure;

FIG. 8 is a schematic of a circuit showing the electrical components for a pressure sensitive device according to an exemplary embodiment of the present disclosure;

FIG. 9 is a schematic of a force to voltage circuit driven by a −5V DC excitation voltage, coupled with a flexible sensor, for use with a pressure sensitive device according to an exemplary embodiment of the present disclosure;

FIG. 10 illustrates a device for limiting movements adverse to health or surgical recovery according to an exemplary embodiment of the present disclosure;

FIG. 11 illustrates a device for limiting movements adverse to health or surgical recovery according to another exemplary embodiment of the present disclosure;

FIG. 12 illustrates a system for limiting movements adverse to health or surgical recovery according to an exemplary embodiment of the present disclosure; and

FIG. 13 illustrates a method for limiting movements adverse to health or surgical recovery according to an exemplary embodiment of the present disclosure; and

FIG. 14 depicts a pressure sensitive device having alternative housing according to an exemplary embodiment of the present disclosure.

### DETAILED DESCRIPTION OF EXEMPLARY EMBODIMENTS

In the following detailed description of exemplary embodiments, reference is made to the accompanying drawings, which form a part hereof, and within which are shown by way of illustration specific embodiments by which the disclosed invention may be practiced. It is to be understood that other embodiments may be utilized and structural changes may be made without departing from the spirit or scope of the invention. As used in this specification and the appended claims, the singular forms “a,” “an”, and “the” include plural referents, unless the content clearly dictates otherwise. As used in this specification and the appended claims, the term “or” is generally employed in its sense including “and/or” unless the context clearly dictates otherwise.

While certain aspects of conventional technologies have been discussed to facilitate disclosure of the invention, applicants in no way disclaim these technical aspects, and it is contemplated that the disclosed invention may encompass one or more of the conventional technical aspects discussed herein. The present disclosure may address one or more of the problems and deficiencies of the prior art discussed above.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

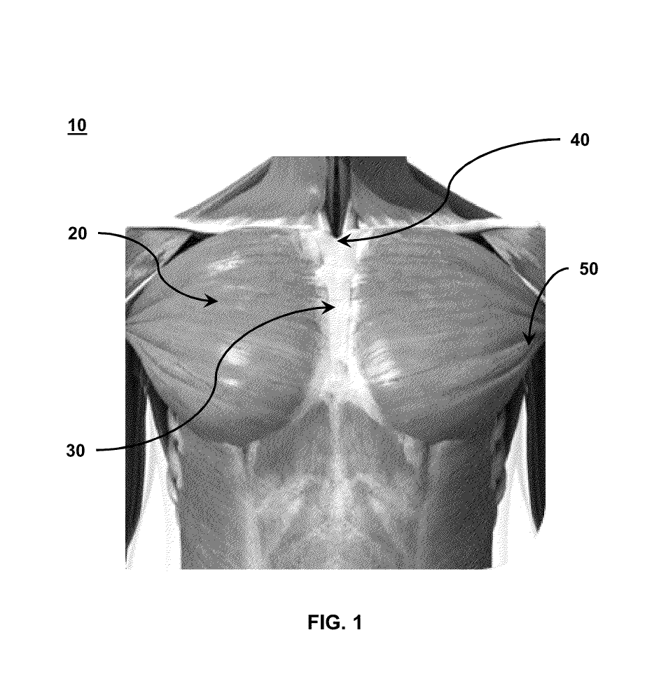

During open-heart surgery, the sternum is cut to allow access to the heart. After surgery, the sternum is again secured using cerclage stainless steel wires, to allow for healing to begin. During recovery from this procedure, the sternum is prone to a variety of stresses that primarily originate from the pectoral muscle group. FIG. 1 is a perspective view of a torso 10 showing the pectoralis major 20, the sternum 30, the area of fixation between the pectoralis major and the sternum 30, and the area of insertion of the sternal head of the pectoralis major 40. FIG. 1 shows the close relationship between the pectoral muscles and the sternum 30, outlining the points of fixation between the muscles and bones.

The pectoralis major 20 is the largest muscle in the chest; and it works synergistically with the shoulder to allow movement of the arms. Each motion generates an associated force; these forces are then transferred from the muscles to the bone, more specifically the sternum. Any tension experienced by the pectoral muscle group will apply tension to the sternum. After open-heart surgery, the sternum becomes vulnerable to the forces associated with everyday activities, such as elevating the arms and using the arms to assist in moving from a sitting to standing (STS) position. During the recovery period (e.g., 6 to 8 weeks), it is important for patients to refrain from such motions. It has been observed, however, that patients often continue to exhibit these motor patterns, putting them at risk of serious complications.

In an exemplary embodiment, the present disclosure provides a reminding device, e.g., a “glove,” which detects applied pressure to the palm and signals a corresponding alarming feature in response to the detected force. Components and aspects of this product may include, but are not limited to, rechargeable, pressure sensing, alarming, maximal sensing area over the palmar region of the hand, minimal coverage of the back of the hand, secured fixation, removable, and washable. The device is generally composed of a durable, deformable material and less deformable material, electronics, pressure sensor(s), and corresponding alarming feature. A possible embodiment of the disclosed product is to be used as a rehabilitation device to remind the user not to engage the assistance of the hands after open heart surgery. However, other possible embodiments may encompass any demographic in which the restriction of pressure applied to the hand or other monitored area may be advisable.

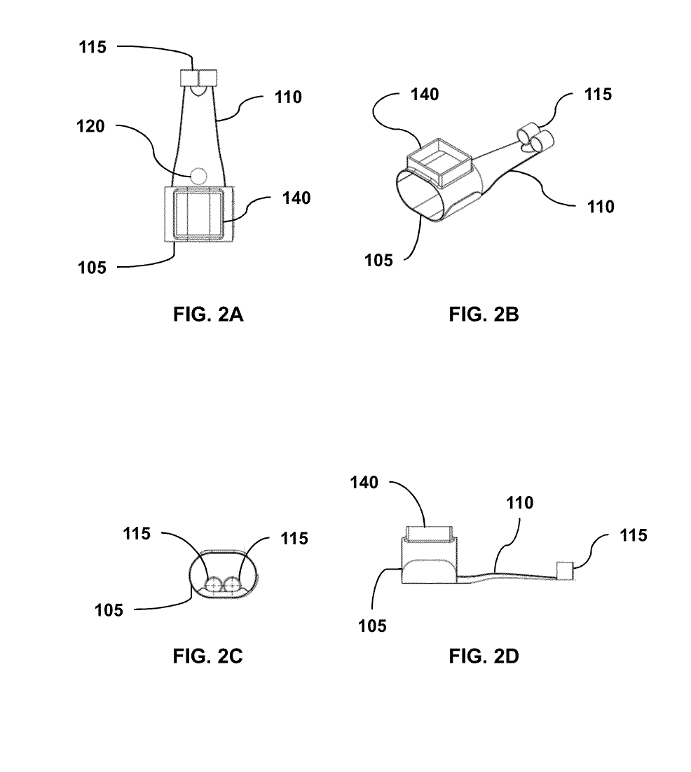

FIGS. 2A-2D depict pressure sensitive devices according to an exemplary embodiment of the present disclosure. FIG. 2A is a top schematic view of a device showing a housing 110 having a sensor 120. FIG. 2B is a schematic view of the device depicted in FIG. 2A showing a front-side perspective. FIG. 2C provides an end view and FIG. 2D provides a side view of the exemplary pressure sensitive device. According to an exemplary embodiment, the disclosed device includes a housing 110 having a flexible force sensor 120 and an indicator component 140. The housing 110 may be wearable on the hand and wrist of a patient, and may include finger-receiving compartments 115.

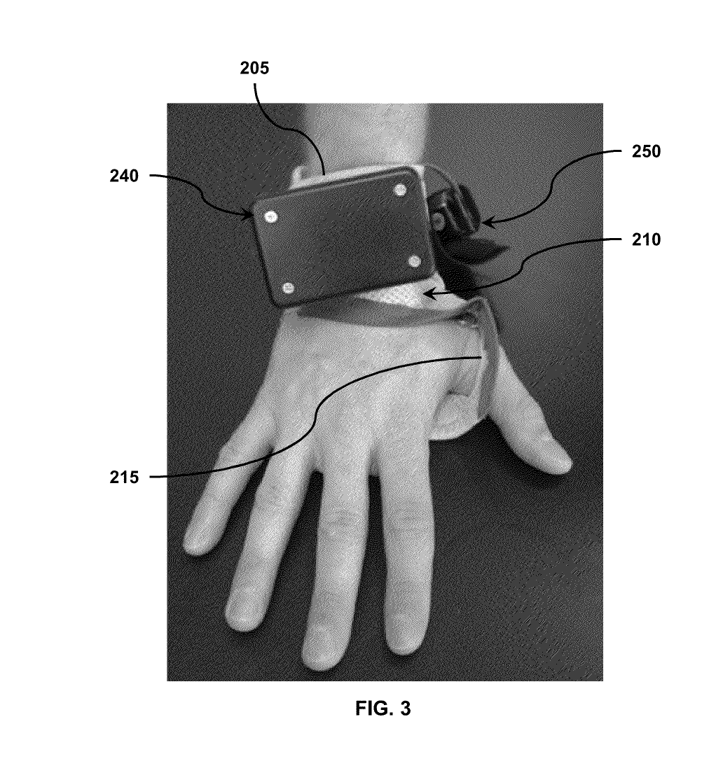

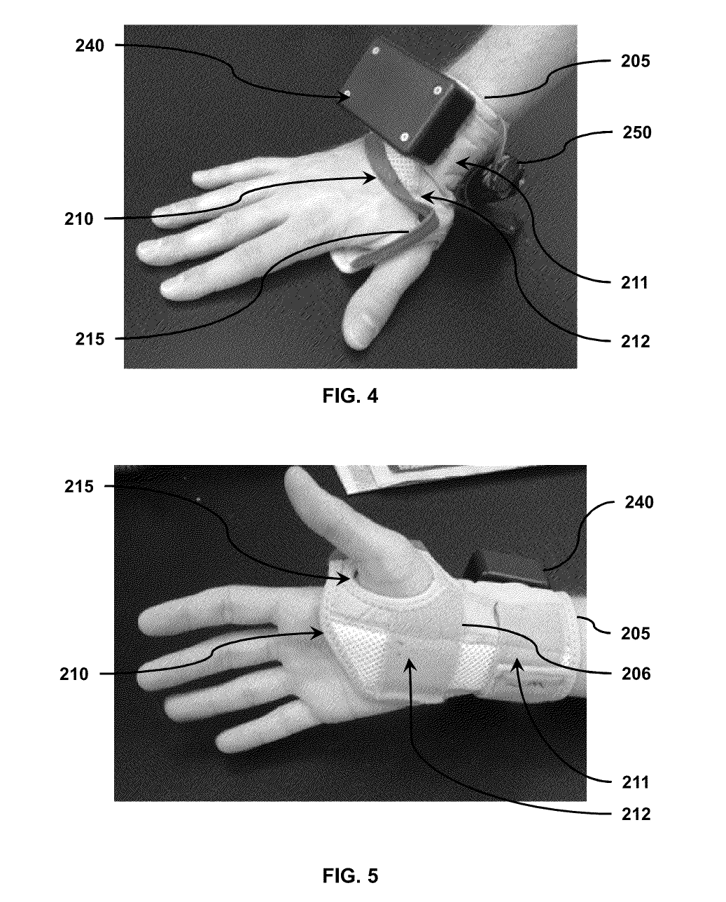

FIGS. 3-5 depict a pressure sensitive device having a glove member according to an exemplary embodiment of the present disclosure. The disclosed glove member 210 is made of thin, lightweight material and is configured and sized so as to allow, in whole or in part, enclosing of a hand. Materials used may include, but are not limited to, cloth, knitted wool, felted wool, leather, rubber, latex, and neoprene. According to an exemplary embodiment, the fabrication material includes comfortable cloth materials.

Glove member 210 includes a wrist portion 211 and a hand portion 212. Wrist portion 211 has a proximate end 205 and a distal end 206. Proximate end 205 forms an opening through which a hand may be inserted, as illustrated in FIGS. 3-5. Wrist portion 211 may extend varying degrees in length between proximate end 205 and distal end 206 of glove member 210. Distal end 206 forms a part of hand portion 212. Hand portion 212 includes a plurality of finger-receiving compartments 215 adapted to receive fingers of a human hand.

According to an exemplary embodiment, hand portion 212 includes a large single opening and a single finger-receiving compartment. In an aspect, five (5) finger-receiving compartments are provided as part of glove member 210. In another aspect, less than five (5) finger-receiving compartments may be present, with a proximal securing feature around the user's wrist. According to another exemplary embodiment, the disclosed device may further include a housing that defines a sensing area, as shown in FIG. 5. The housing may include self-adhesive material, bandage wrap, etc., to facilitate securement relative to a desired anatomical location and/or region. While varying degrees of material may be used, it is an objective of the present disclosure to use minimal materials in order to increase user compliance and discomfort with wearing the device.

FIGS. 3-5 illustrate an exemplary embodiment of the present disclosure engaged with a human hand. Hand portion 212 along the dorsal aspect of a human hand is substantially left open. A separate thumb-receiving compartment 215 extends over the dorsal aspect of thumb, as illustrated in FIGS. 3-5.

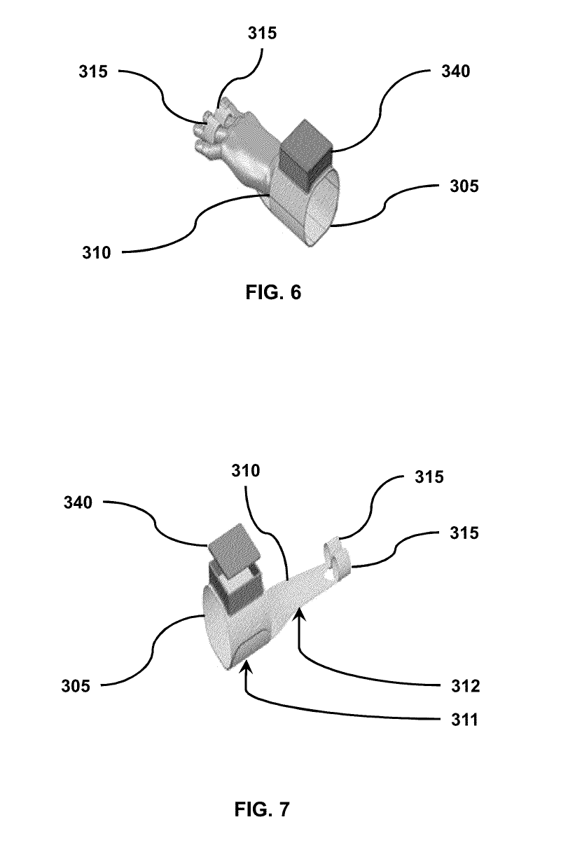

FIGS. 6-7 illustrate a pressure sensitive device according to another exemplary embodiment. The housing or glove member 310 includes a plurality of finger-receiving compartments 315, adapted to engage a human hand, that extend from palmer aspect of glove member 315 to wrap around fingers, according to an exemplary embodiment of the present disclosure. Dorsal aspect of a human hand is substantially left open when engaged according to this exemplary embodiment.

Palmer aspect of hand portion 312 should substantially cover a palm region of a human hand. FIG. 5 illustrates a device engaged along palmer aspect of a human hand according to an exemplary embodiment of the present disclosure. At least one attachment element extends along palmer aspect of glove member 310. For example, fixed placement of glove member may be achieved using strap members engaging one another through an attachment mechanism. For example, the attachment mechanism may include a hook and loop fastener system, e.g., VELCRO®. The straps can vary in width and location to provide differing degrees of support and comfort throughout the healing process. According to an exemplary embodiment, a strap may be located along palmer aspect of hand portion 312 and another strap may extend along anterior aspect of wrist portion 311.

Referring back to FIG. 2A, a force sensor 120 to measure a load applied to the extremity is attached with respect to an anterior aspect of wrist portion. FIG. 2A illustrates the location of the force sensor in accordance with an exemplary embodiment of the present disclosure. Referring back to FIG. 5, a force sensor may be placed along the anterior aspect of wrist portion 212 to detect application of improper force. Locating the sensor in the wrist region allows detection of typical force applied when a patient is moving from a STS position. Most individuals push down on an armrest or object to assist in standing. That force can further injure a patient after heart surgery. According to an exemplary embodiment, the force sensor may be flexible in design and may take the form of a piezoelectric sensor.

Piezoelectric sensors have a proven history of versatility and reliability in measuring pressures and forces. According to exemplary embodiments, applicable sensors include Model #A401 FlexiForce Standard and Force Load commercially available from Tekscan, Inc. (South Boston, Mass.). This sensor model, which is depicted in FIG. 9 along with an exemplary circuit configuration, has a thickness of about 0.208 mm, a length of about 56.8 mm, and a width of about 31.8 mm. The approximate sensing area is 25.4 mm in diameter and the substrate material is composed of polyester. The connecting component of the Model #A401 sensor includes 2 male square pins. A wide range of forces are measurable with this exemplary sensor. For example, loads from 0-7000 lbs. may be measured using the exemplary circuitry disclosed herein. Though not necessary, it is noted that this force range can be extended by reducing the drive voltage, V_{T}, or resistance value of the feedback resistor, R_{F}. Conversely, sensitivity can be increased for measurement of lower forces by increasing V_{T} or R_{F}. Of note, the force sensor should be selected to operate in the applicable force range (based on clinical application) and be adapted to measure force threshold(s) depending on risks associated with patient recovery.

It is foreseeable that various other force sensors may be utilized to achieve similar results. Sensors used should be resilient to twisting, tension, and wet conditions to ensure the device will continue to perform as intended and to determine the expected life of the product.

According to another exemplary embodiment, an indicator component 240 is attached along the posterior aspect of wrist portion 211 as illustrated in FIGS. 3-5. The indicator component 240 is in electrical communication with the force sensor. The indicator component may include an audible alarm, a vibration, or other types of visual, aural, and/or tactile alert notifications.

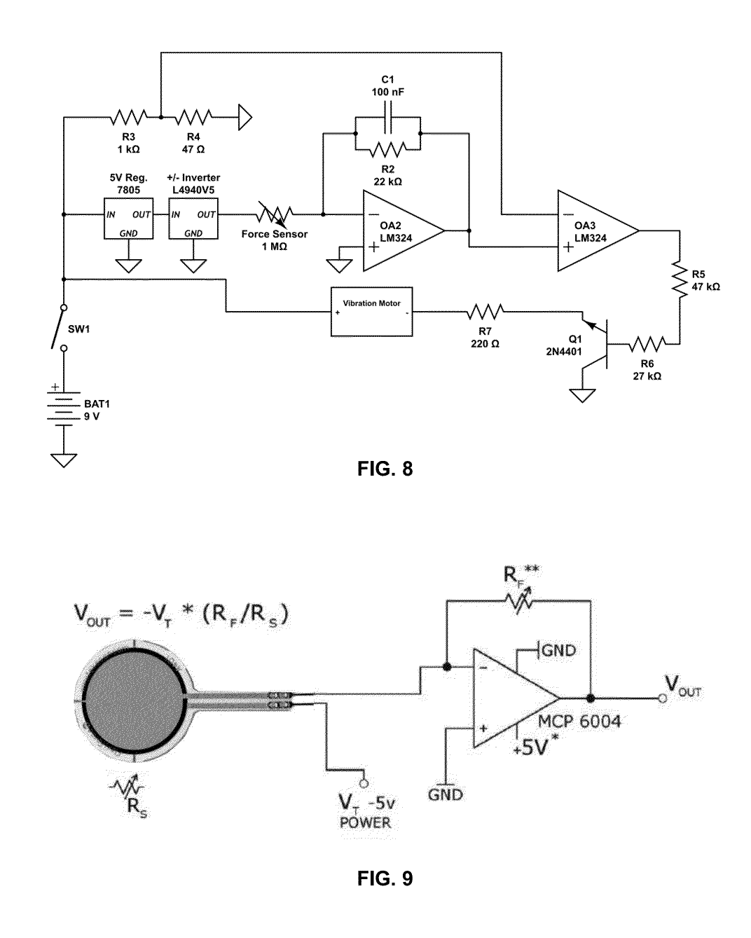

FIG. 8 illustrates electronic components in accordance with an exemplary embodiment of the present disclosure. The disclosed force sensor detects movements in a hand that may approach and/or represent potentially restricted movements. A signal is transmitted to a vibration motor in response to potentially restricted movements. According to an exemplary embodiment of the present disclosure, the vibration motor is activated to signal/indicate that a restricted movement is occurring. A battery or alternative power source is located in electrical communication with the vibration motor and force sensor.

According to another exemplary embodiment, an audible alarm may be included. An external alarm system may detect when the force sensor exceeds a predetermined and pre-defined amount of force. Once the threshold is met (or exceeded), an audible alarm is activated to indicate that the user should stop the present movement. The disclosed audible alarm may be in combination with, or in place of, the vibration motor. The audible alarm can be used to alert staff and family members as well.

The pressure sensing threshold is the value that must be met or exceeded to trigger the alert feature of the disclosed device, system or method. The pressure sensing threshold is generally proportional to the resistance in the electrical circuit, which may be varied to address specific clinical applications, positioning modalities and/or user criteria. Thus, the disclosed pressure sensing threshold is selected based on the conditions under which it is to operate, and may be adjusted to meet specific user and/or clinical needs.

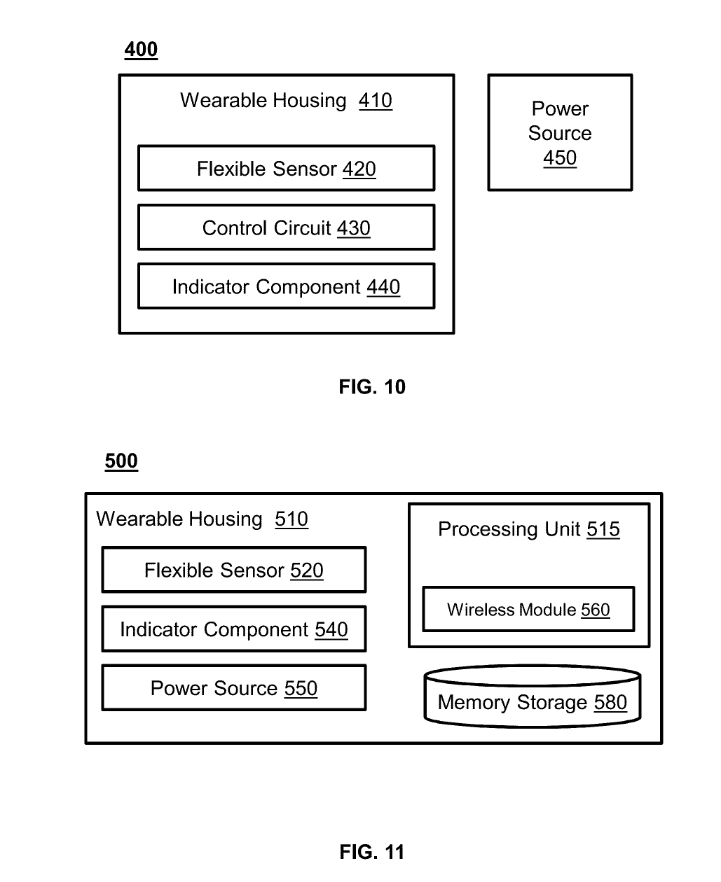

FIG. 10 illustrates a device 400 according to an exemplary embodiment of the present disclosure. The device 400 includes a wearable housing 410 having a sensor 420, a control circuit 430, and an indicator component 440. The sensor 420 and the indicator component 440 may be electronically coupled via the control circuit 430 to enable communication. The device 400 may also include a power source 450 that is electronically coupled to the sensor 420 and indicator component 440. The power source 450 provides power to the device components. In an aspect, the power source 450 includes a battery. The battery may be rechargeable. In another aspect, the power source 450 includes a battery electronically coupled to a solar cell for recharging the battery.

The indicator component 440 may include a vibration motor or a speaker and is adapted to generate an alert to the patient and/or other caregiver. The alert may include an audible alert, a vibration, or other type of aural, visual and/or tactile notification.

FIG. 11 illustrates a device 500 according to a further exemplary embodiment of the present disclosure. The device includes a wearable housing 510 that includes a sensor 520, an indicator component 540, and a power source 550. Device 500 also includes a processing unit 515 having a wireless module 560 and a memory storage device 580. The sensor 520, indicator component 540, power source 550, processing unit 515, and memory storage 580 are electronically coupled to enable system communications.

The processing unit 515 may be changeably programmable to enable user input and/or adjustment of the predefined threshold criteria. The wireless module 560 is adapted to receive and transmit wireless communications to the cloud or a remote computing device. The device 500 may store data in a memory storage 580 (e.g., a memory chip) and/or in the cloud by transmitting the data via the wireless module 560. Data stored in the memory storage 580 may be uploaded to the cloud manually or at predetermined intervals and/or may be retained on the device 500.

The wireless module 560 enables remote input of user-defined criteria such as the predefined threshold for force applied. The wireless module 560 further enables review of recorded data by patients, caregivers, and other healthcare professionals to further aid the rehabilitation process. According to another aspect, the processing unit 515, upon receiving a pressure value exceeding the predefined threshold, may simultaneously signal the indicator component 540 and the wireless module 560. Thus, as the indicator component 540 generates an alert for the patient/user, the wireless module 560 may transmit a “caregiver” alert to a respective caregiver (e.g., a family member or a healthcare professional).

According to another aspect, an accelerometer may also warn against undesirable or restricted movements. The accelerometer may be used in conjunction with or as an alternative to the force sensor. According to an exemplary embodiment, both an accelerometer and a force sensor are used, each with a predetermined and pre-defined threshold.

The adjusting and programing of desired thresholds can be set in a variety of methods, which include but are not limited to, manual adjustments via programmable buttons, remote programing via Wi-Fi (e.g., smartphone, computer, tablet, etc.) or the like.

The disclosed device is advantageously capable of being programed with safety features that may include, but are not limited to, moisture detecting shut off capabilities, remote monitoring, integration with clinical equipment for data transmission, integration with recreational Wi-Fi detecting equipment for data transmission, remote threshold programing and locking of threshold to reduce the risk of tampering.

Predetermined thresholds may be determined through the creation of unique patient profiles. Unique patient profiles may be generated by recording movements by a skilled clinician that are dangerous to perform after certain surgeries. Those profiles may be stored on a computer based server. A memory chip on the pressure sensitive device can be used to load a particular patient's profile.

Predetermined thresholds change based upon specific patient profile and adapted to patient's needs. For example, a patient recovering from heart surgery should avoid moving from a STS position. This requires pressure sensitive device to detect a pressure threshold relating to injuring the sternum or chest area. However, a patient recovering from shoulder surgery would require a different patient profile. Shoulder recovery may allow more pressure to be applied when moving from the STS position, but may require a reduction in arm movement as detected by the accelerometer.

The device or system may relay information to remote locations, which may include but is not limited to any Wi-Fi sensing device such as smart phones, tablets, computers, nursing stations, and similar technologies. For example, the device may be a “dumb” device that transmits its output via Bluetooth to a computing device. Computing device may include, but is not limited to, a computer based server, mobile device such as a smartphone, tablet computing device, laptop computer and desktop computer.

A memory chip may also be used to log and track patient's actions while wearing the pressure sensitive device. Data may be reviewed and accessed by health care personnel for use in assistance of rehabilitation and treatment. Device can log and alert the end user and a remote health care worker of specific activities. An inactivity profile may sense the lack of movement or pressure actuation and fire an event to have a wellness check performed.

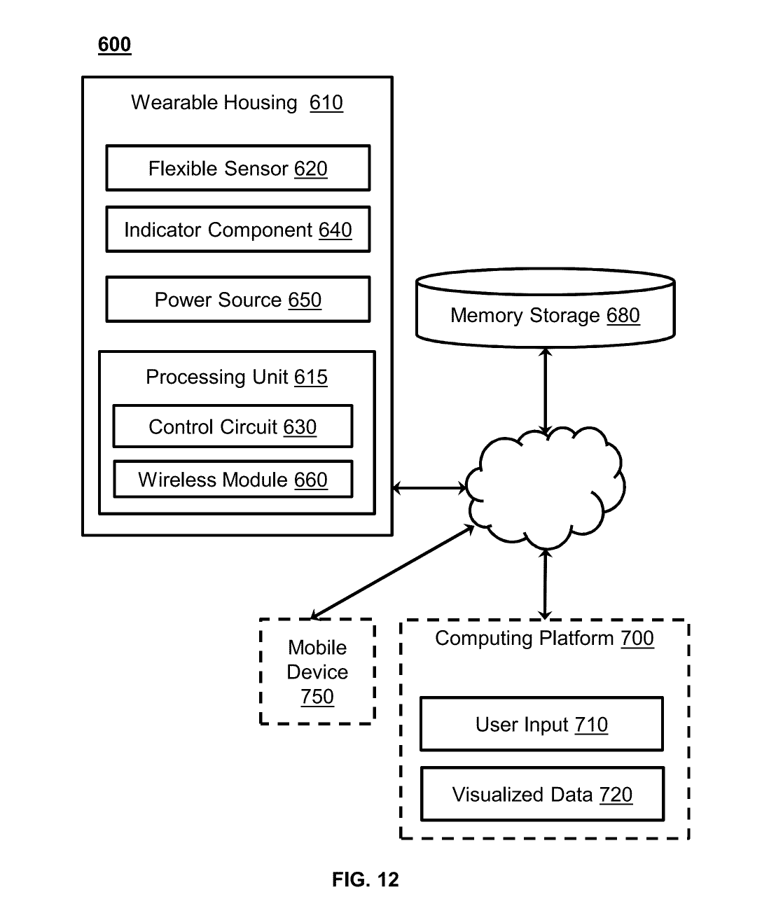

FIG. 12 depicts a system according to an exemplary embodiment of the present disclosure. The system includes a wearable housing 610 that includes sensor 620, an indicator component 640, and a power source 650; a processing unit 615 having a control circuit 630, and a wireless module 660 for transmitting and receiving wireless communications. The disclosed system may be adapted to connect to a computing platform 700, such as a desktop computer or mobile device 750, and/or may be further adapted to connect to external memory storage 680. The computing platform may include a display for user input 710, e.g., for input of predefined threshold(s) for pressure value, and for visualizing patient data recorded and logged from a device, i.e., visualized data 720.

In an aspect, the system connects to the memory storage device and/or the computing platform 700 or mobile device 750 via the cloud. In another aspect, the system is electronically coupled with one or more of the memory storage or computing platform.

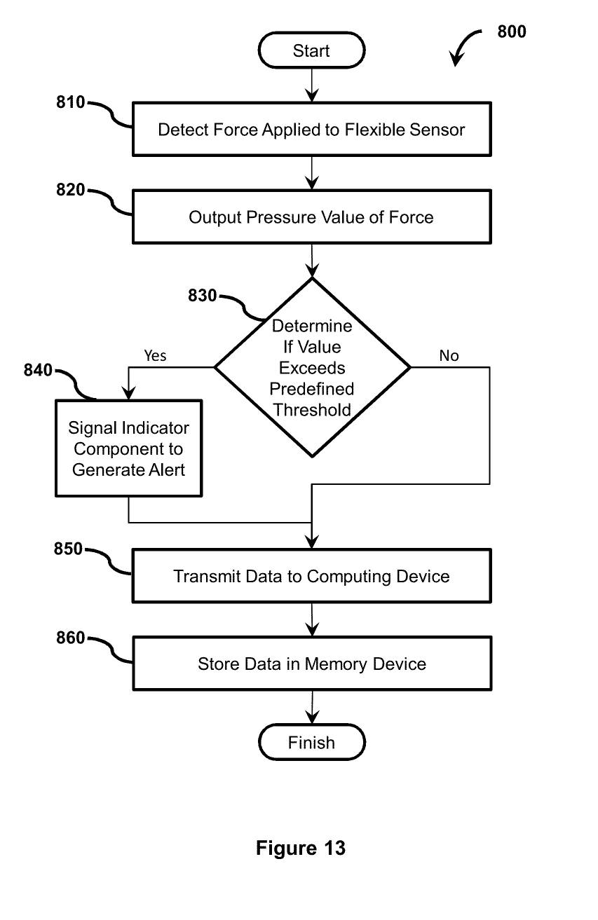

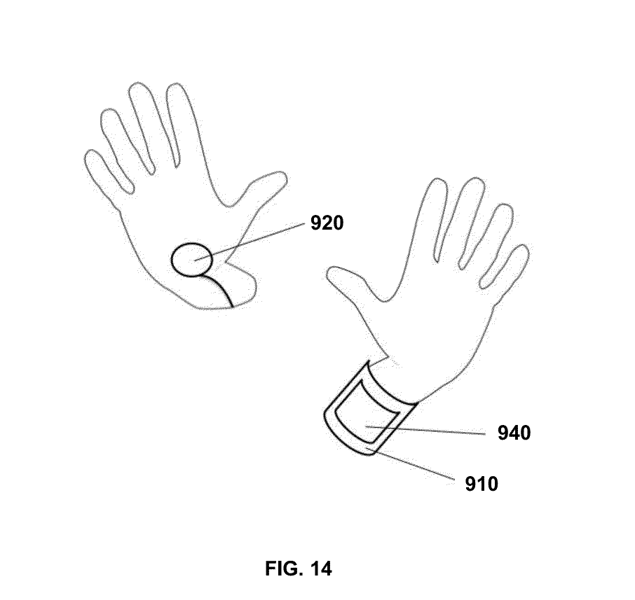

FIG. 13 illustrates a method 800 for limiting movement adverse to health or surgical recovery. The method includes detecting a force applied at the site of sensor 810; outputting a pressure value of the force 820; determining if the pressure value exceeds a predefined threshold 830 and, if so, signaling an indicator component to generate an alert 840; transmitting data to a computing device 850; and storing data in a memory device 860.

The disclosed device is not limited to a housing that takes the form of a glove. More specifically, the disclosed housing for supporting the operative components of the disclosed device/system may take various physical and structural forms. For example, operative components of the disclosed device/system may be supported by such physical arrangements as self-adhesive materials, bandage, wire, mesh, and manual fixation, among others.

FIG. 14 illustrates a perspective view of a right hand with an alternative housing supporting operative components according to an exemplary embodiment of the present disclosure. In an aspect, the sensor 920 is attached to an anterior aspect of the right hand via an adhesive material. The indicator component 940 is attached with respect to the posterior aspect of the right hand (or wrist as depicted in FIG. 14) using an adhesive material or band 910.

The advantages set forth above, and those made apparent from the foregoing description, are efficiently attained. Since certain changes may be made in the above construction without departing from the scope of the disclosure, it is intended that all matters contained in the foregoing description or shown in the accompanying drawings shall be interpreted as illustrative and not in a limiting sense.

What is claimed is:

1. A pressure sensitive device for detecting force in connection with a health condition, comprising:
a wearable housing;
at least one sensor disposed with respect to the wearable housing and configured to detect a pressure value;
an indicator component configured to generate an alert; and
a control circuit electronically coupled to the at least one sensor and the indicator component,
wherein the control circuit is configured to activate the indicator component when the pressure value exceeds a predefined threshold.

2. The pressure sensitive device of claim 1, further comprising a power source coupled with the flexible sensor and the indicator component.

3. The pressure sensitive device of claim 2, wherein the power source includes a battery.

4. The pressure sensitive device of claim 3, wherein the battery is rechargeable.

5. The pressure sensitive device of claim 3, further comprising a solar power cell electronically coupled with the battery for recharging the battery.

6. The pressure sensitive device of claim 1, wherein the control circuit is changeably programmable for input of the predefined threshold.

7. The pressure sensitive device of claim 1, further comprising a memory device coupled with the control circuit for recording pressure values.

8. The pressure sensitive device of claim 1, further comprising a wireless module for wirelessly transmitting and receiving data.

9. The pressure sensitive device of claim 8, wherein the wireless module includes Bluetooth technology.

10. The pressure sensitive device of claim 1, wherein the indicator component includes a vibration motor.

11. The pressure sensitive device of claim 1, wherein the alert includes at least one of an audible alert, a visual alert or a tactile alert.

12. The pressure sensitive device of claim 1, further comprising an accelerometer configured to activate the indicator component upon exceeding a second predefined threshold.

13. The pressure sensitive device of claim 1 wherein the wearable housing includes a self-adhesive material.

14. A pressure sensitive device for detecting movement adverse to surgical recovery, comprising:
a wearable housing including a hand portion having a plurality of compartments adapted to receive at least one finger of a human hand, and a wrist portion having a proximate end and a distal end, the proximate end defining an opening and the distal end forming a continuous part of the hand portion;
a sensor positioned along the anterior aspect of the wrist portion; and
an indicator component electronically coupled with the sensor,
wherein the indicator component generates an alert when the pressure value exceeds a predefined threshold.

15. The pressure sensitive device of claim 14, further comprising at least one of a memory device or a wireless module for recording data.

16. A pressure sensitive system for detecting and recording movement adverse to rehabilitation, comprising:
a sensor configured to detect a pressure value;
an indicator component configured to generate an alert; and
a processing unit electronically coupled with the sensor and the indicator component, and having a wireless module for transmitting and receiving wireless communications;
wherein the processing unit is configured to activate the indicator component when the pressure value exceeds a predefined threshold, and wherein the wireless module wirelessly transmits the pressure value for data storage.

17. The system of claim 16, wherein the processing unit is changeably programmable for input of the predefined threshold.

18. The system of claim 17, wherein the input of the predefined threshold is wirelessly received by the wireless module.

19. A method of detecting movements adverse to recovery, comprising:
detecting a force applied to a sensor;
outputting a pressure value of the force to a processing unit; and
signaling an indicator component to generate an alert if the pressure value exceeds a predefined threshold.

20. The method of claim 19, further comprising at least one of transmitting the pressure value to a computing device, and storing the pressure value in a memory device.
